(19) United States
(12) Patent Application Publication
(10) Pub. No.: US 2020/0076000 A1
(43) Pub. Date: Mar. 5, 2020

(54) NON-AQUEOUS ELECTROLYTE AND NON-AQUEOUS ELECTROLYTE SECONDARY BATTERY

(71) Applicant: Panasonic Intellectual Property Management Co., Ltd., Osaka-shi, Osaka (JP)

(72) Inventors: Mitsuru Iwai, Osaka (JP); Yuanlong Zhong, Osaka (JP)

(73) Assignee: Panasonic Intellectual Property Management Co., Ltd., Osaka-shi, Osaka (JP)

(21) Appl. No.: 16/495,698

(22) PCT Filed: Jan. 23, 2018
(86) PCT No.: PCT/JP2018/001860

§ 371 Date: Sep. 19, 2019

(30) Foreign Application Priority Data
Mar. 23, 2017 (JP) 2017-058206

(51) Int. Cl. H01M 10/0569; H01M 4/525; H01M 10/0525; C07D 309/10; C07D 319/12

(52) U.S. Cl. CPC H01M 10/0569; H01M 4/525; H01M 2004/028; C07D 309/10; C07D 319/12; H01M 10/0525

(57) ABSTRACT

A non-aqueous electrolyte includes a lithium salt, and a non-aqueous solvent dissolving the lithium salt. The non-aqueous solvent contains a fluorinated cyclic carbonic acid ester, a carboxylic anhydride A having a structure represented by a general formula (1) below, and a carboxylic anhydride B having a structure represented by a general formula (2) below. (In the general formula (1), n represents 0 or 1, and R₁ to R₄ each independently represent a hydrogen atom, an alkyl group, an alkenyl group, or an aryl group.) (In the general formula (2), R₅ to R₈ each independently represent a hydrogen atom, an alkyl group, an alkenyl group, or an aryl group.)

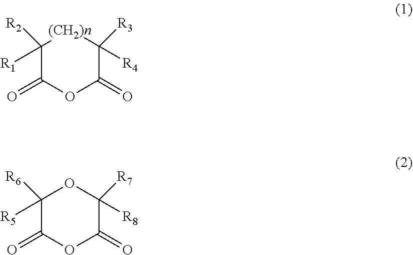

### TECHNICAL FIELD

The present invention relates to an improvement of the non-aqueous electrolyte in non-aqueous electrolyte secondary batteries.

### BACKGROUND ART

Non-aqueous electrolyte secondary batteries represented by lithium ion secondary batteries include a positive electrode, a negative electrode, and a non-aqueous electrolyte. The non-aqueous electrolyte contains a lithium salt and a non-aqueous solvent dissolving the lithium salt. Aiming for improvement of battery performance, various studies have been made on the components of the non-aqueous electrolyte.

Patent Literature 1 discloses a non-aqueous electrolyte containing a fluorinated cyclic carbonic acid ester, such as fluoroethylene carbonate, in the non-aqueous solvent. During charging, at the negative electrode, reductive decomposition of the fluorinated cyclic carbonic acid ester occurs, forming a coating layer (Solid Electrolyte Interface: SEI) on the surface of a negative electrode active material. The coating layer thus formed improves the charge-discharge cycle characteristics at room temperature.

Patent Literature 2 proposes to add succinic anhydride and diglycolic anhydride to a non-aqueous electrolyte containing propylene carbonate, so that a coating layer is formed on the surface of a negative electrode active material, and thereby the reductive decomposition of the propylene carbonate at the negative electrode can be inhibited.

### CITATION LIST

### Patent Literature

[PTL 1] Japanese Patent Laid-Open No. 2013-182807
[PTL 2] Japanese Patent Laid-Open No. 2005-078866

### SUMMARY OF INVENTION

A coating layer derived from a fluorinated cyclic carbonic acid ester lacks thermal stability. Therefore, in a high temperature environment, the coating layer is destroyed. As a result, decomposition of the non-aqueous electrolyte proceeds during charging and discharging, and in association therewith, an increase in gas generation, a rise in internal resistance due to side reaction products, and a decrease in battery capacity occur.

In view of the above, one aspect of the present disclosure relates to a non-aqueous electrolyte including a lithium salt, and a non-aqueous solvent dissolving the lithium salt. The non-aqueous solvent contains a fluorinated cyclic carbonic acid ester, a carboxylic anhydride A having a structure represented by a general formula (1) below, and a carboxylic anhydride B having a structure represented by a general formula (2) below.

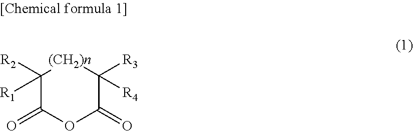

In the general formula (1), n represents 0 or 1, and R₁ to R₄ each independently represent a hydrogen atom, an alkyl group, an alkenyl group, or an aryl group.

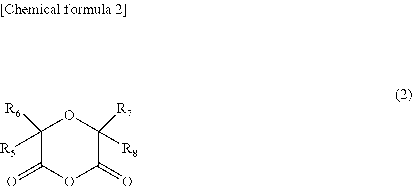

In the general formula (2), R₅ to R₈ each independently represent a hydrogen atom, an alkyl group, an alkenyl group, or an aryl group.

Another aspect of the present disclosure relates to the non-aqueous electrolyte secondary battery including the aforementioned non-aqueous electrolyte, a positive electrode, and a negative electrode.

With the non-aqueous electrolyte according to the present disclosure, when the non-aqueous solvent contains a fluorinated cyclic carbonic acid ester, the high-temperature storage characteristics of the non-aqueous electrolyte secondary battery can be improved.

### BRIEF DESCRIPTION OF DRAWING

FIG. 1 A partially cut-away oblique view of a non-aqueous electrolyte secondary battery according to an embodiment of the present invention.

### DESCRIPTION OF EMBODIMENTS

A non-aqueous electrolyte according to an embodiment of the present invention includes a lithium salt, and a non-aqueous solvent dissolving the lithium salt. The non-aqueous solvent contains a fluorinated cyclic carbonic acid ester, a carboxylic anhydride A having a structure represented by a general formula (1) below, and a carboxylic anhydride B having a structure represented by a general formula (2) below.

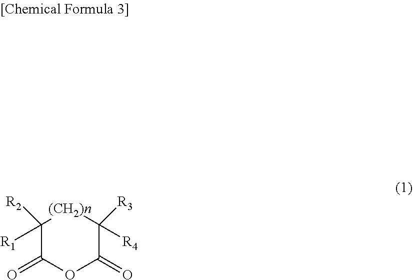

In the general formula (1), n represents 0 or 1, and R₁ to R₄ each independently represent a hydrogen atom, an alkyl group, an alkenyl group, or an aryl group.

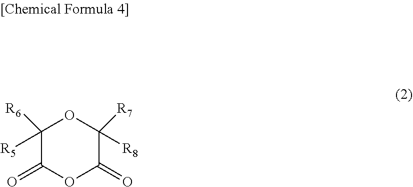

In the general formula (2), R₅ to R₈ each independently represent a hydrogen atom, an alkyl group, an alkenyl group, or an aryl group.

By adding the carboxylic anhydride A and the carboxylic anhydride B to a non-aqueous electrolyte containing a fluorinated cyclic carbonic acid ester, the surface of a negative electrode active material is coated with an SEI layer having lithium-ion conductivity and being thermally and chemically stable. This can suppress destruction of the coating layer in a high temperature environment, and avoid unfavorable results from decomposition of the non-aqueous electrolyte following the destruction of the coating layer, such as an increase in gas generation, a decrease in battery capacity, and a rise in internal resistance (coating layer resistance).

By adding the carboxylic anhydride A and the carboxylic anhydride B to a non-aqueous electrolyte containing a fluorinated cyclic carbonic acid ester, the surface of a positive electrode active material is also coated with a layer that inhibits reaction with the non-aqueous electrolyte. Even when the positive electrode active material is a lithium-containing transition metal oxide containing nickel, this can suppress gas generation and decrease in battery capacity that occur due to reactions between the non-aqueous electrolyte and the positive electrode active material in a high temperature environment.

(Fluorinated Cyclic Carbonic Acid Ester)

The fluorinated cyclic carbonic acid ester contains at least one fluorine atom in its molecule. The fluorinated cyclic carbonic acid ester preferably has a structure represented by a general formula (3) below.

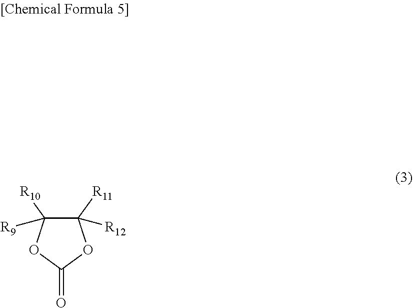

In the general formula (3), R₉ to R₁₂ each independently represent a hydrogen atom, a fluorine atom, an alkyl group, or a fluorinated alkyl group, and at least one of R₉ to R₁₂ is a fluorine atom or a fluorinated alkyl group. The number of carbon atoms in the alkyl group or the fluorinated alkyl group is preferably 1 to 3. Preferably, R₉ to R₁₂ each independently represent a hydrogen atom or a fluorine atom, and at least one of R₉ to R₁₂ is a fluorine atom. Fluoroethylene carbonate is more preferred among them.

The amount of the fluorinated cyclic carbonic acid ester in the non-aqueous solvent is preferably 0.1 to 50 vol %. The amount of the fluorinated cyclic carbonic acid ester as herein referred to is a volume percentage in the whole non-aqueous solvent, except for the carboxylic anhydride A and the carboxylic anhydride B.

The amount of the fluorinated cyclic carbonic acid ester in the non-aqueous solvent can be determined by, for example, gas chromatography-mass spectrometry (GC/MS).

(Carboxylic Anhydride A)

The carboxylic anhydride A has a structure represented by the general formula (1) above. In the general formula (1), n represents 0 or 1, and R₁ to R₄ each independently represent a hydrogen atom, an alkyl group, an alkenyl group, or an aryl group. The number of carbon atoms in the alkyl group or the alkenyl group is preferably 1 to 20. The aryl group is, for example, a phenyl group, a benzyl group, a tolyl group, or a xylyl group, and is preferably a phenyl group. The carboxylic anhydride A is preferably at least one of succinic anhydride and glutaric anhydride.

In view of improving the high-temperature storage characteristics and the initial characteristics of the battery, the amount of the carboxylic anhydride A in the non-aqueous electrolyte is preferably 0.1 to 2.0 mass %, more preferably 0.5 to 1.5 mass %.

(Carboxylic Anhydride B)

The carboxylic anhydride B has a structure represented by the general formula (2) above. In the general formula (2), R₅ to R₈ each independently represent a hydrogen atom, an alkyl group, an alkenyl group, or an aryl group. The number of carbon atoms in the alkyl group or the alkenyl group is preferably 1 to 20. The aryl group is, for example, a phenyl group, a benzyl group, a tolyl group, or a xylyl group, and is preferably a phenyl group. Preferable examples of the carboxylic anhydride B include diglycolic anhydride, methyldiglycolic anhydride, dimethyldiglycolic anhydride, ethyldiglycolic anhydride, methoxydiglycolic anhydride, ethoxydiglycolic anhydride, vinyldiglycolic anhydride, allyldiglycolic anhydride, and divinyldiglycolic anhydride. These may be used singly or in combination of two or more kinds. In view of suppressing rise in internal resistance of the battery in a high temperature environment, the carboxylic anhydride B is more preferably diglycolic anhydride.

In view of improving the high-temperature storage characteristics and the initial characteristics of the battery, the amount of the carboxylic anhydride B in the non-aqueous electrolyte is preferably 0.1 to 2.0 mass %, more preferably 0.5 to 1.5 mass %.

The mass ratio of the carboxylic anhydride A to the carboxylic anhydride B in the non-aqueous electrolyte is preferably 1:1/6 to 1:6. In this case, the decrease in battery capacity after storage at high temperatures, the rise in internal resistance after storage at high temperatures, and the gas generation during storage at high temperatures can be suppressed in a well-balanced manner. In view of suppressing decrease in battery capacity after storage at high temperatures, the above mass ratio is preferably 1:1 to 1:3, more preferably 1:1. Furthermore, in view of suppressing increase in internal resistance after storage at high temperatures and gas generation during storage at high temperatures, the above mass ratio is more preferably not 1:1, more preferably 1:1/6 to 1:1/3, and 1:3 to 1:6.

The amounts of the carboxylic anhydride A and the carboxylic anhydride B in the non-aqueous electrolyte can be determined by, for example, gas chromatography-mass spectrometry (GC/MS).

(Non-Aqueous Solvent)

Examples of the non-aqueous solvent include: in addition to the fluorinated cyclic carbonic acid ester and the carboxylic anhydrides A and B, a cyclic carbonic acid ester, such as propylene carbonate (PC) and ethylene carbonate (EC); a chain carbonic acid ester, such as diethyl carbonate (DEC), ethyl methyl carbonate (EMC) and dimethyl carbonate (DMC); and a cyclic carboxylic acid ester, such as γ-butyrolactone (GBL) and γ-valerolactone. These may be used singly or in combination of two or more kinds.

The non-aqueous electrolyte may contain an additive, so that better charge-discharge characteristics of the battery can be achieved. Examples of the additive include vinylene carbonate (VC), vinyl ethylene carbonate, cyclohexylbenzene (CHB), and fluorobenzene. The amount of the additive in the non-aqueous electrolyte is, for example, 0.01 to 15 mass %, and may be 0.05 to 10 mass %.

(Lithium Salt)

Examples of the lithium salt include LiPF₆, LiBF₄, LiClO₄, LiAsF₆, LiCF₃SO₃, and imide salts, such as LiN(SO₂F)₂ (abb.: LiFSI) and LiN(SO₂CF₃)₂ (abb.: LiTFSI). In view of lithium-ion conductivity, the lithium salt preferably includes at least one selected from the group consisting of LiPF₆, LiFSI, and LiTFSI. These lithium salts may be used singly or in combination of two or more kinds.

The concentration of the lithium salt in the non-aqueous electrolyte is, for example, 0.5 to 2 mol/L.

A non-aqueous electrolyte secondary battery according to an embodiment of the present invention includes the aforementioned non-aqueous electrolyte, a positive electrode including a positive electrode active material, and a negative electrode including a negative electrode active material. By using the non-aqueous electrolyte as described above, the high-temperature storage characteristics of the battery can be improved.

(Positive Electrode)

The positive electrode includes, for example, a positive electrode current collector, and a positive electrode mixture layer formed on a surface of the positive electrode current collector. The positive electrode mixture layer can be formed by applying a positive electrode slurry comprising a positive electrode mixture dispersed in a dispersion medium, onto a surface of the positive electrode current collector, and drying the slurry. The dry applied film may be rolled, if necessary. The positive electrode mixture contains a positive electrode active material as an essential component, and may contain optional components, such as a binder, an electrically conductive agent, and a thickener.

The positive electrode active material may be, for example, a lithium-containing transition metal oxide. Examples of the lithium-containing transition metal oxide include LiₐM_{b}O_{c}, LiMPO₄, and Li₂MPO₄F. Here, M is at least one element selected from the group consisting of Na, Mg, Sc, Y, Mn, Fe, Co, Ni, Cu, Zn, Al, Cr, Pb, Sb, and B. a=0 to 1.2, b=0.1 to 1.0, and c=2.0 to 4.0. The value a representing the molar ratio of lithium is a value upon production of the active material and subjected to increase and decrease during charging and discharging.

In view of achieving a higher capacity, the lithium-containing transition metal oxide preferably contains Ni. Preferred among the lithium-containing transition metal oxides containing Ni is LiₐNiₓCo_{y}Al_{z}O₂ (where 0≤a≤1.2, 0.8≤x≤1.0, 0≤y≤0.2, 0≤z≤0.1, and x+y+z=1). By containing Ni with x being 0.8 or more, it is possible to achieve a higher capacity. By containing Co with y being 0.2 or less, it is possible to increase the stability of the crystal structure of the lithium-containing transition metal oxide, while maintaining a high capacity. By containing Al with z being 0.1 or less, it is possible to enhance the thermal stability of the lithium-containing transition metal oxide, while maintaining the output characteristics.

The binder may be a resin material, examples of which include: fluorocarbon resin, such as polytetrafluoroethylene and polyvinylidene fluoride (PVDF); polyolefin resin, such as polyethylene and polypropylene; polyamide resin, such as aramid resin; polyimide resin, such as polyimide and polyamide-imide; acrylic resin, such as polyacrylic acid, polymethyl acrylate, and ethylene-acrylic acid copolymer; vinyl resin, such as polyacrylnitrile and polyvinyl acetate; polyvinyl pyrrolidone; polyether sulfone; and a rubbery material, such as styrene-butadiene copolymer rubber (SBR). These may be used singly or in combination of two or more kinds.

Examples of the conductive agent include: graphite, such as natural graphite and artificial graphite; carbon blacks, such as acetylene black; conductive fibers, such as carbon fibers and metal fibers; fluorinated carbon; metal powders, such as aluminum; conductive whiskers, such as zinc oxide and potassium titanate; conductive metal oxides, such as titanium oxide; and organic conductive materials, such as phenylene derivatives. These may be used singly or in combination of two or more kinds.

Examples of the thickener include: carboxymethyl cellulose (CMC) and modified products thereof (including salts such as Na salt); cellulose derivatives (e.g., cellulose ether), such as methyl cellulose; saponificated products of a polymer having a vinyl acetate unit, such as polyvinyl alcohol; polyether (e.g., polyalkylene oxide, such as polyethylene oxide). These may be used singly or in combination of two or more kinds.

Examples of the positive electrode current collector include a non-porous electrically conductive base material (e.g., metal foil), and a porous electrically conductive base material (e.g., mesh, net, punched sheet). The positive electrode current collector may be made of, for example, stainless steel, aluminum, an aluminum alloy, and titanium. The positive electrode current collector may have any thickness, and is, for example, 3 to 50 μm thick.

Although not particularly limited, examples of the dispersion medium include: water; alcohols, such as ethanol; ethers, such as tetrahydrofuran; amides, such as dimethylformamide; N-methyl-2-pyrrolidone (NMP); and a mixed solvent of these.

(Negative Electrode)

The negative electrode includes, for example, a negative electrode current collector, and a negative electrode mixture layer formed on a surface of the negative electrode current collector. The negative electrode mixture layer can be formed by applying a negative electrode slurry comprising a negative electrode mixture dispersed in a dispersion medium, onto a surface of the negative electrode current collector, and drying the slurry. The dry applied film may be rolled, if necessary. The negative electrode mixture layer may be formed on one surface or both surfaces of the negative electrode current collector. The negative electrode mixture contains a negative electrode active material as an essential component, and may contain optional components, such as a binder, an electrically conductive agent, and a thickener. Examples of the binder, the thickener, and the dispersion medium are as those exemplified for the positive electrode. Examples of the conductive agent are as those exemplified for the positive electrode, except graphite.

The negative electrode active material includes, for example, a carbon material that electrochemically absorbs and releases lithium ions. Examples of the carbon material include graphite, easily graphitizable carbon (soft carbon), and non-graphitizable carbon (hard carbon). Preferred among them is graphite, which is excellent in stability during charging and discharging and has small irreversible capacity. Graphite means a material having a graphite-type crystal structure, examples of which include natural graphite, artificial graphite, graphitized mesophase carbon particles. The carbon material may be used singly or in combination of two or more kinds.

Examples of the negative electrode current collector include a non-porous electrically conductive base material (e.g., metal foil), and a porous electrically conductive base material (e.g., mesh, net, punched sheet). The negative electrode current collector may be made of, for example, stainless steel, nickel, a nickel alloy, copper, and a copper alloy. The negative electrode current collector may have any thickness. In view of the balance between strength and weight savings of the negative electrode, the thickness is preferably 1 to 50 more preferably 5 to 20 μm.

In an exemplary structure of the non-aqueous electrolyte secondary battery, an electrode group formed by winding the positive electrode and the negative electrode with the separator interposed therebetween is housed together with the non-aqueous electrolyte in an outer case. The wound-type electrode group may be replaced with a different form of the electrode group, for example, a stacked-type electrode group formed by stacking the positive electrode and the negative electrode with the separator interposed therebetween. The non-aqueous electrolyte secondary battery may be in any form, such as cylindrical type, prismatic type, coin type, button type, or laminate type.

(Separator)

Usually, it is desirable to interpose a separator between the positive electrode and the negative electrode. The separator is excellent in ion permeability and has moderate mechanical strength and electrically insulating properties. The separator may be, for example, a microporous thin film, a woven fabric, or a nonwoven fabric. The separator is preferably made of, for example, polyolefin, such as polypropylene or polyethylene.

A detailed description will be given below of each component other than the negative electrode, with a prismatic wound-type battery taken as an example. It is to be noted, however, that the type, shape, and other features of the non-aqueous electrolyte secondary battery are not particularly limited.

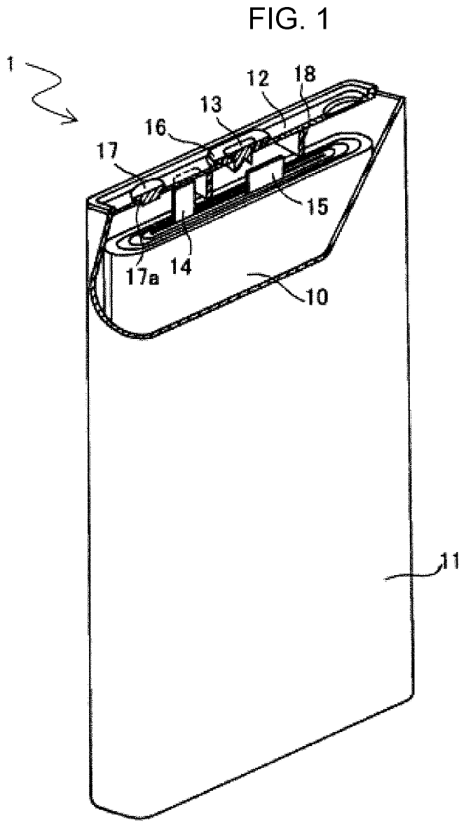

FIG. 1 is a partially cut-away oblique view of a non-aqueous electrolyte secondary battery according to an embodiment of the present invention. In FIG. 1, a non-aqueous electrolyte secondary battery 1 is illustrated partially cut away, to show the configuration of an essential part thereof. A prismatic battery case 11 houses a flat wound-type electrode group 10 and the aforementioned non-aqueous electrolyte (not shown).

The electrode group 10 is formed by winding a sheet-like positive electrode and a sheet-like negative electrode, with a separator interposed between the positive and negative electrodes. To the positive electrode current collector of the positive electrode included in the electrode group 10, a positive electrode lead 14 is connected at its one end. The positive electrode lead 14 is connected at its other end to a sealing plate 12 serving as a positive electrode terminal. A negative electrode lead 15 is connected at its end to the negative electrode current collector, and at its other end, connected to a negative electrode terminal 13 provided approximately at the center of the sealing plate 12. A gasket 16 is disposed between the sealing plate 12 and the negative electrode terminal 13, to provide electrical insulation therebetween. A frame member 18 made of an electrically insulating material is disposed between the sealing plate 12 and the electrode group 10, to provide electrical insulation between the negative electrode lead 15 and the sealing plate 12. The sealing plate 12 is joined to the open end of the prismatic battery case 11, and seals the prismatic battery case 11. The sealing plate 12 has an injection port 17a. The non-aqueous electrolyte is injected into the prismatic battery case 11 through the injection port 17a. Thereafter, the injection port 17a is closed with a sealing cap 17.

### EXAMPLES

The present invention will be specifically described below with reference to Examples and Comparative Examples. The present invention, however, is not limited to the following Examples.

### Example 1

(1) Production of Positive Electrode

LiNi_{0.8}Co_{0.15}Al_{0.05}O₂ serving as a positive electrode active material, acetylene black, and polyvinylidene fluoride were mixed in a mass ratio of 100:1:1. The mixture was added with N-methyl-2-pyrrolidone (NMP), and then stirred in a mixer (T.K. HIVIS MIX, available from PRIMIX Corporation), to prepare a positive electrode slurry. Next, the positive electrode slurry was applied onto aluminum foil. The applied films were dried, and then rolled, to give a positive electrode with a positive electrode mixture layer having a density of 3.6 g/cm³ on both sides of the aluminum foil.

(2) Production of Negative Electrode

Graphite powder (average particle size: 20 μm), sodium carboxymethyl cellulose (CMC-Na), and styrene-butadiene rubber (SBR) were mixed in a mass ratio of 100:1:1. The mixture was added with water, and stirred in a mixer (T.K. HIVIS MIX, available from PRIMIX Corporation), to prepare a negative electrode slurry. Next, the negative electrode slurry was applied onto copper foil. The applied films were dried, and then rolled, to give a negative electrode with a negative electrode mixture layer having a density of 1.6 g/cm³ formed on both sides of the copper foil.

(3) Preparation of Non-Aqueous Electrolyte

A mixed solvent containing fluoroethylene carbonate (FEC), dimethyl carbonate (DMC), and ethyl methyl carbonate (EMC) at a volume ratio of 15:40:45 at room temperature was prepared. To the resultant mixed solvent, LiPF₆ serving as a lithium salt was dissolved at a concentration of 1.3 mol/L, to prepare a non-aqueous electrolyte. To the non-aqueous electrolyte, succinic anhydride (SA) and diglycolic anhydride (DGA) were added. The amount of the succinic anhydride (SA) in the non-aqueous electrolyte was 0.5 mass %. The amount of the diglycolic anhydride (DGA) in the non-aqueous electrolyte was 0.5 mass %.

(4) Fabrication of Non-Aqueous Electrolyte Secondary Battery (Laminate-Type Battery)

The positive electrode and the negative electrode, with a tab attached to each electrode, were wound spirally with a separator interposed therebetween such that the tabs were positioned at the outermost layer, thereby to form an electrode group. The separator was a 20-μm-thick polyethylene microporous film. The electrode group was inserted into an outer case made of aluminum laminate film and dried under vacuum at 105° C. for 2 hours. The non-aqueous electrolyte was then injected into the outer case, and the opening of the case was sealed. A non-aqueous electrolyte secondary battery (design capacity: 50 mAh) was thus obtained.

### Comparative Example 1

A non-aqueous electrolyte secondary battery was fabricated in the same manner as in Example 1, except that neither SA nor DGA was added to the non-aqueous electrolyte.

### Comparative Example 2

A non-aqueous electrolyte secondary battery was fabricated in the same manner as in Example 1, except that the amount of SA in the non-aqueous electrolyte was 0.5 mass %, and no DGA was added to the non-aqueous electrolyte.

### Comparative Example 3

A non-aqueous electrolyte secondary battery was fabricated in the same manner as in Example 1, except that the amount of DGA in the non-aqueous electrolyte was 0.5 mass %, and no SA was added to the non-aqueous electrolyte.

The batteries of Example and Comparative Examples were evaluated for the following properties.

[Evaluation]

(A) Capacity Recovery Ratio After High-Temperature Storage

<Charging>

In a 25° C. environment, the batteries were subjected to a constant-current charging at a current of 0.5 It until the voltage reached 4.1 V, and then to a constant-voltage charging at a voltage of 4.1 V until the current reached 0.05 It. After charging, the batteries were left to stand for 10 minutes.

<Discharging>

After left to stand, in a 25° C. environment, the batteries were subjected to a constant-current discharging at a current of 0.5 It until the voltage reached 3.0 V, to obtain a discharge capacity C1 (initial capacity).

Batteries were prepared separately. The batteries were charged under the same conditions as those in (A) above, and then stored for 15 days in a 45° C. environment. After storage, the batteries were discharged under the same conditions as those in (A) above, and further charged and discharged under the same conditions as those in (A) above, to obtain a discharge capacity C2 (recovery capacity).

Then, a capacity recovery ratio was determined from the following equation.

Capacity recovery ratio (%)=(Discharge capacity C2/Discharge capacity C1)×100

(B) Internal Resistance (DC-IR) Change Ratio After High-Temperature Storage

The batteries fabricated above were, in a 25° C. environment, charged at a constant current of 0.3 It until the battery voltage reached 4.1 V, and then discharged at a constant current of 0.5 It for 10 seconds. From the change in voltage before and after the discharge and the discharge current value, a DC resistance (1st-day resistance value) was determined.

Thereafter, the batteries were stored for 15 days in a high temperature environment of 45° C. After storage, the batteries were left to stand for 1 hour in a 25° C. environment. Thereafter, a DC resistance (15th-day resistance value) was determined in a similar method as above.

Then, an internal resistance change ratio was determined from the following equation.

Internal resistance change ratio (%)=(15th-day resistance value/1st-day resistance value)×100

(C) Gas Generation Amount During High-Temperature Storage

The batteries were charged under the same conditions as those in (A) above, and then put into water. From the change in water level, a volume of each battery before storage was determined. Batteries were prepared separately. The batteries were charged under the same conditions as those in (A) above, and then stored for 15 days in a 45° C. environment. With respect to the batteries after storage, a volume of each battery was determined in a similar manner as above. From the change in volume of the battery before and after storage, a gas generation amount was determined.

The gas generation amount was expressed as an index, with the gas generation amount of the battery of Comparative Example 1 taken as 100.

The evaluation results of (A) to (C) are shown in Table 1. Batteries were rated as having favorable high-temperature storage characteristics when they exhibited a higher capacity recovery ratio, a lower internal resistance change ratio, and a smaller gas generation amount than those of the battery of Comparative Example 1.

TABLE 1

| | | | Evaluation of high-temperature | | |
|---|---|---|---|---|---|
| | SA amount in | DGA amount in | storage characteristics | | |
| | non-aqueous | non-aqueous | Capacity | Internal resistance | Gas generation |
| | electrolyte | electrolyte | recovery ratio | change ratio | amount |
| | (mass %) | (mass %) | (%) | (%) | (index) |
| Com. Ex. 1 | 0 | 0 | 95.2 | 140.6 | 100.0 |
| Com. Ex. 2 | 0.5 | 0 | 97.6 | 160.3 | 81.9 |
| Com. Ex. 3 | 0 | 0.5 | 95.9 | 135.2 | 113.8 |
| Ex. 1 | 0.5 | 0.5 | 98.0 | 124.5 | 75.7 |

In the battery of Example 1 using an FEC-containing non-aqueous electrolyte added with SA and DGA, as compared to the batteries of Comparative Examples 1 to 3, the capacity recovery ratio was high, the internal resistance change ratio was low, and the gas generation amount was small, showing that excellent high-temperature characteristics were obtained.

In the battery of Comparative Example 2 added with no DGA, as compared to the battery of Comparative Example 1, the internal resistance change ratio was considerably increased. In the battery of Comparative Example 3 added with no SA, as compared to the battery of Comparative Example 1, the gas generation amount was considerably increased.

### Example 2

A non-aqueous electrolyte secondary battery was fabricated and evaluated in the same manner as in Example 1, except that the amount of DGA in the non-aqueous electrolyte was 1.5 mass %.

### Example 3

A non-aqueous electrolyte secondary battery was fabricated and evaluated in the same manner as in Example 1, except that the amount of SA in the non-aqueous electrolyte was 1.5 mass %.

### Example 4

A non-aqueous electrolyte secondary battery was fabricated and evaluated in the same manner as in Example 1, except that the amount of SA in the non-aqueous electrolyte was 0.5 mass %, and the amount of DGA in the non-aqueous electrolyte was 3.0 mass %.

### Example 5

A non-aqueous electrolyte secondary battery was fabricated and evaluated in the same manner as in Example 1, except that the amount of DGA in the non-aqueous electrolyte was 0.5 mass %, and the amount of SA in the non-aqueous electrolyte was 3.0 mass %.

The evaluation results are shown in Table 2.

TABLE 2

| | | | Evaluation of high-temperature | | |
|---|---|---|---|---|---|
| | SA amount in | DGA amount in | storage characteristics | | |
| | non-aqueous | non-aqueous | Capacity | Internal resistance | Gas generation |
| | electrolyte | electrolyte | recovery ratio | change ratio | amount |
| | (mass %) | (mass %) | (%) | (%) | (index) |
| Ex. 1 | 0.5 | 0.5 | 98.0 | 124.5 | 75.7 |
| Ex. 2 | 0.5 | 1.5 | 97.7 | 115.9 | 65.8 |
| Ex. 3 | 1.5 | 0.5 | 95.8 | 109.2 | 48.9 |
| Ex. 4 | 0.5 | 3 | 96.9 | 92.9 | 51.4 |
| Ex. 5 | 3 | 0.5 | 96.5 | 95.5 | 39.9 |

In the batteries of Examples 2 to 5, too, in which SA and DGA were added to an FEC-containing non-aqueous electrolyte, the capacity recovery ratio was high, the internal resistance change ratio was low, and the gas generation amount was small as compared to the battery of Comparative Example 1, showing that excellent high-temperature characteristics were obtained. In the batteries of Examples 2 to 5 in which SA and DGA were added in different amounts, as compared to the battery of Example 1 in which SA and DGA were added in equal amounts, the internal resistance change ratio and the gas generation amount were further suppressed small. In the battery of Example 1 in which SA and DGA were added in equal amounts, as compared to the batteries of Examples 2 to 5 in which SA and DGA were added in different amounts, the capacity recovery ratio was high.

Table 3 below shows the evaluation results of the initial characteristics of Examples 1 to 5. The discharge capacity in Table 3 shows the discharge capacity Cl determined in (A) above. The internal resistance in Table 3 shows the 1st-day resistance value determined in (B) above. The discharge capacity and the internal resistance value in Table 3 are each expressed as an index, with the discharge capacity and the internal resistance value of Example 1 taken as 100, respectively.

TABLE 3

| | | | Evaluation of initial | |
|---|---|---|---|---|
| | SA amount in | DGA amount in | characteristics | |
| | non-aqueous | non-aqueous | Discharge | Internal |
| | electrolyte | electrolyte | capacity | resistance |
| | (mass %) | (mass %) | (index) | (index) |
| Ex. 1 | 0.5 | 0.5 | 100.0 | 100.0 |
| Ex. 2 | 0.5 | 1.5 | 98.1 | 115.7 |
| Ex. 3 | 1.5 | 0.5 | 100.3 | 122.6 |
| Ex. 4 | 0.5 | 3 | 87.0 | 254.0 |
| Ex. 5 | 3 | 0.5 | 97.4 | 192.2 |

The batteries of Examples 1 to 3 exhibited, in the initial state, a larger discharge capacity and a lower internal resistance than those of the batteries of Examples 4 and 5. In view of the initial characteristics, the amount of each carboxylic anhydride in the non-aqueous electrolyte is preferably 0.1 to 2.0 mass %. In the battery of Example 1 in which SA and DGA were added in equal amounts, as compared to the batteries of Examples 2 to 5 in which SA and DGA were added in different amounts, the internal resistance in the initial characteristics was low.

### INDUSTRIAL APPLICABILITY

The non-aqueous electrolyte secondary battery of the present invention is useful as a main power source for mobile communication devices, portable electronic devices, and others.

### REFERENCE SIGNS LIST

1: non-aqueous electrolyte secondary battery

10: wound-type electrode group

11: prismatic battery case

12: sealing plate

13: negative electrode terminal

14: positive electrode lead

15: negative electrode lead

16: gasket

17: sealing cap

17a: injection port

18: frame member

1. A non-aqueous electrolyte, comprising
a lithium salt, and a non-aqueous solvent dissolving the lithium salt,
the non-aqueous solvent containing a fluorinated cyclic carbonic acid ester, a carboxylic anhydride A having a structure represented by a general formula (1) below, and a carboxylic anhydride B having a structure represented by a general formula (2) below. 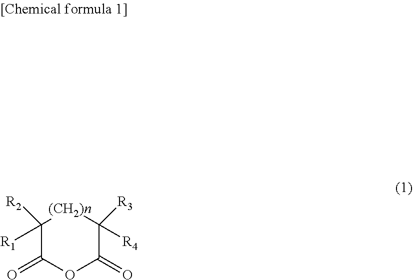
(In the general formula (1), n represents 0 or 1, and R₁to R₄ each independently represent a hydrogen atom, an alkyl group, an alkenyl group, or an aryl group.) 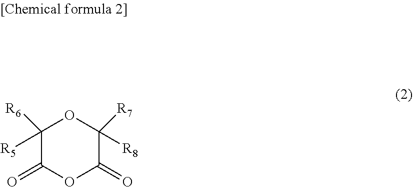
(In the general formula (2), R₅ to R₈ each independently represent a hydrogen atom, an alkyl group, an alkenyl group, or an aryl group.)

2. The non-aqueous electrolyte of claim 1, wherein an amount of the fluorinated cyclic carbonic acid ester in the non-aqueous solvent is 0.1 to 50 vol %.

3. The non-aqueous electrolyte of claim 1, wherein an amount of the carboxylic anhydride A in the non-aqueous electrolyte is 0.1 to 2.0 mass %.

4. The non-aqueous electrolyte of claim 1, wherein an amount of the carboxylic anhydride B in the non-aqueous electrolyte is 0.1 to 2.0 mass %.

5. The non-aqueous electrolyte of claim 1, wherein the amount of the carboxylic anhydride B in the non-aqueous electrolyte is greater than the amount of the carboxylic anhydride A in the non-aqueous electrolyte.

6. The non-aqueous electrolyte of claim 1, wherein the amount of the carboxylic anhydride A in the non-aqueous electrolyte is greater than the amount of the carboxylic anhydride B in the non-aqueous electrolyte.

7. The non-aqueous electrolyte of claim 1, wherein the carboxylic anhydride A includes at least one selected from the group consisting of succinic anhydride and glutaric anhydride.

8. The non-aqueous electrolyte of claim 1, wherein the carboxylic anhydride B includes at least one selected from the group consisting of diglycolic anhydride, methyldiglycolic anhydride, dimethyldiglycolic anhydride, ethyldiglycolic anhydride, methoxydiglycolic anhydride, ethoxydiglycolic anhydride, vinyldiglycolic anhydride, allyldiglycolic anhydride, and divinyldiglycolic anhydride.

9. The non-aqueous electrolyte of claim 1, wherein the fluorinated cyclic carbonic acid ester includes fluoroethylene carbonate.

10. A non-aqueous electrolyte secondary battery, comprising the non-aqueous electrolyte of claim 1, a positive electrode, and a negative electrode.

11. The non-aqueous electrolyte secondary battery of claim 10, wherein the positive electrode includes a lithium-containing transition metal oxide containing Ni.
